(19) United States
(12) Patent Application Publication
(10) Pub. No.: US 2012/0078348 A1
(43) Pub. Date: Mar. 29, 2012

(54) APPARATUS AND METHODS FOR DELIVERING AT LEAST ONE THERAPEUTIC AGENT

(76) Inventor: David E. Orr, Piedmont, SC (US)

(21) Appl. No.: 13/322,344

(22) PCT Filed: May 27, 2010
(86) PCT No.: PCT/US10/36420

§ 371 Date: Dec. 20, 2011

Related U.S. Application Data
(60) Provisional application No. 61/181,956, filed on May 28, 2009.

(51) Int. Cl. A61F 2/82

(52) U.S. Cl. 623/1.42

(57) ABSTRACT

The present invention provides apparatus and methods for treating tissue by delivering at least one therapeutic agent to the tissue. In one embodiment, the apparatus comprises first and second membranes (30,32) in sealing engagement with strut segments (25,26) of at least one stent. A first membrane pocket is disposed between the first and second membranes(30,32), and a first therapeutic agent is disposed within the first membrane pocket. In other embodiments, the first and second membranes (130, 140) may be disposed between first and second spaced apart stents (114, 122), or only a single membrane may be provided. In each instance, the quantity of the therapeutic agent delivered is not limited by the surface area of the stent struts or the lumen diameter of the struts.

### TECHNICAL FIELD

The present invention relates to apparatus and a method for delivering at least one therapeutic agent to a patient. In the preferred embodiments the apparatus is in the form of an implantable medical device.

### BACKGROUND ART

There are several instances in which it may become desirable to introduce therapeutic agents into a human or animal body. For example, therapeutic drugs, bioactive materials or cells may be introduced to achieve a biological effect. The biological effect may include an array of targeted results, such as inducing homeostasis, reducing restenosis likelihood, or treating cancerous tumors or other diseases, or supplementing physiologically deficient bioprocesses.

Many of such therapeutic agents are injected using an intravenous (IV) technique and via oral medicine. While such techniques permit the general introduction of medicine, in many instances it may be desirable to provide localized or targeted delivery of therapeutic agents, which may allow for the guided and precise delivery of agents to selected target sites.

For example, localized delivery of therapeutic agents to a tumor may reduce the exposure of the therapeutic agents to normal, healthy tissues, which may reduce potentially harmful side effects. Similarly, therapeutic agents may be delivered locally to a diseased portion of a coronary vessel to reduce, halt or reverse the progression of a stenosis, or may be delivered to a diseased portion of the aorta in order to reduce, halt or reverse the progression of an abdominal aortic aneurysm.

Drug eluting stents have shown great promise in treating various diseases, such as coronary artery disease, by helping to deliver therapeutic agents to perform an intended function such as restoring blood flow in arteries and reducing restenosis rates. Typically, a therapeutic agent is coated onto the struts of the stent, or alternatively, the agent may be injected into a lumen of a stent strut and then released through one or more pores in the strut that are in communication with the lumen.

While drug eluting stents achieve a beneficial localized delivery of a desired therapeutic agent, limitations exist with respect to current designs. For example, it may be difficult to deliver multiple different therapeutic agents simultaneously to a desired target site. Moreover, the quantity of the therapeutic agents delivered is generally limited by the surface area of the stent struts or the lumen diameter of the struts, and attempts to increase these stent features to deliver a greater quantity of the therapeutic agents may adversely affect stent characteristics, for example, by increasing the stent's profile or reducing its flexibility.

### DISCLOSURE OF THE INVENTION

The present invention seeks to provide improved apparatus and an improved method for delivering therapeutic agents to a patient.

According to an aspect of the present invention, there is provided apparatus for delivering at least one therapeutic agent to a target site, as specified in claim 1.

According to another aspect of the present invention, there is provided a method of delivering at least one therapeutic agent to a target site, as specified in claim 11.

According to another aspect of the present invention, there is provided apparatus for delivering at least one therapeutic agent to a target site, as specified in claim 17.

There are taught herein systems and methods for treating tissue by delivering at least one therapeutic agent to the tissue. In each embodiment, the quantity of the therapeutic agent delivered is not limited by the surface area of the stent struts or the lumen diameter of the struts.

In a first embodiment, a system for delivering at least one therapeutic agent comprises at least one stent having first and second strut segments. A first membrane has a first region that is in sealing engagement with the first strut segment, and further has a second region in sealing engagement with the second strut segment. Similarly, a second membrane has a first region that is in sealing engagement with the first strut segment, and further has a second region in sealing engagement with the second strut segment. A first membrane pocket is disposed between the first and second membranes, and a first therapeutic agent is disposed within the first membrane pocket.

When the stent is in an expanded state, the first membrane is positioned adjacent to an inner wall of a bodily passageway, while the second membrane is positioned adjacent to a lumen of the passageway. In other words, the first membrane is positioned at an abluminal side and the second membrane is positioned at a luminal side.

The first and/or second membranes may be designed to release the therapeutic agent over a predetermined period of time, and may have the same or different characteristics relative to one another. Moreover, the system further may comprise a second membrane pocket, separate and discrete from the first membrane pocket, and a second therapeutic agent disposed within and delivered via the second membrane pocket.

Advantageously, the system facilitates localized delivery of one or more therapeutic agents to a desired target site. Moreover, the quantity of the therapeutic agents delivered is not limited by the surface area of the stent struts or the lumen diameter of the struts. Rather, an enhanced quantity of the agent may be delivered via one or more membrane pockets disposed between stent struts. Therefore, it is not necessary to increase the surface area of the stent struts or the lumen diameter of the struts to deliver a greater quantity of one or more therapeutic agents, and consequently the stent profile and flexibility is not compromised.

In another embodiment, first and second stents may be longitudinally spaced apart from one another. The first membrane may have a proximal region sealingly engaged with strut segments of the first stent and a distal region sealingly engaged with strut segments of the second stent. Similarly, the second membrane may have a proximal region sealingly engaged with strut segments of the first stent and a distal region sealingly engaged with strut segments of the second stent. Thus, in this embodiment, the membrane pocket is generally disposed between two spaced apart stents.

In yet another embodiment, a system for delivering at least one therapeutic agent to a target site comprises a stent and a membrane, where the membrane is folded over to form a membrane pocket that comprises two proximal boundaries, a distal boundary, and first and second regions formed therebetween. The two proximal boundaries are in sealing engagement with strut segments of the stent. A therapeutic agent is disposed within the membrane pocket, which may form a generally tear-drop shape.

It is to be appreciated that the features of the various embodiments described herein may be combined with one another, for instance to form a medical device having different types of agent pockets or pouches, within the same device.

### BRIEF DESCRIPTION OF THE DRAWINGS

Embodiments of the present invention are described below, by way of example only, with reference to the accompanying drawings, in which:

FIGS. 1-2 are side views of a system for delivering at least one therapeutic agent, in accordance with a first embodiment, shown in contracted and expanded states, respectively;

FIG. 3 is an enlarged view of a portion of the system of FIG. 2;

FIG. 4 is a cross-sectional view along line A-A of FIG. 3;

FIG. 5 is a perspective view illustrating an exemplary membrane that may be used with the system of FIG. 1;

FIG. 6 is a side view of another embodiment for delivering at least one therapeutic agent, as shown in an expanded state;

FIG. 7 is a top view of the system of FIG. 6;

FIG. 8 is a cross-sectional view along line B-B of FIG. 6;

FIG. 9 is a side view of another embodiment for delivering at least one therapeutic agent, as shown in an expanded state;

FIG. 10 is a top view of the system of FIG. 9; and

FIG. 11 is a cross-sectional view along line C-C of FIG. 9.

The components in the Figures are not necessarily to scale, emphasis instead being placed upon illustrating the principles of the teachings herein.

### DESCRIPTION OF THE PREFERRED EMBODIMENTS

In the present application, the term “proximal” refers to a direction that is generally closest to the heart during a medical procedure, while the term “distal” refers to a direction that is furthest from the heart during a medical procedure.

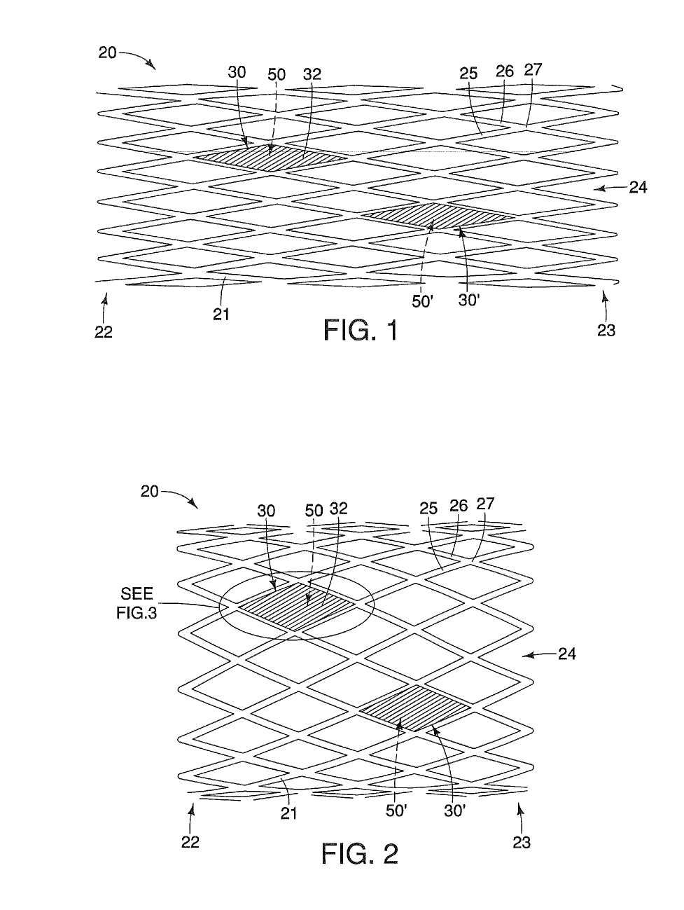

Referring to FIGS. 1-4, a first embodiment of a system 20 for delivering at least one therapeutic agent is described. The system 20 comprises a stent 21 having proximal and distal ends 22 and 23, and a lumen 24 extending therebetween. In this embodiment, the exemplary stent 21 comprises a “Palmaz” type stent having first and second elongate members 25 and 26 that cross at intersections 27, thereby forming a plurality of generally diamond-shaped segments. The stent 21, which is shown in a contracted delivery state in FIG. 1 and an expanded state in FIG. 2, may be balloon expandable or self-expanding. As will be explained in further detail below, a variety of stent configurations may be used in conjunction with the membrane pockets described herein, and the “Palmaz” type stent 21 is merely one illustrative type of stent.

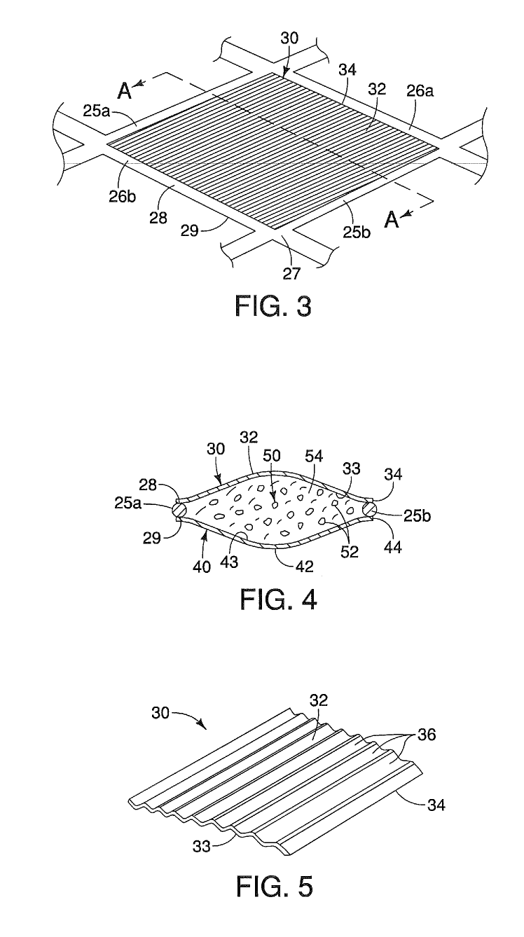

The system 20 comprises at least one membrane pocket 50 suitable for delivering a therapeutic agent 52, as best seen in FIG. 4 below. The membrane pocket 50 is formed between a first membrane 30 and a second membrane 40. As shown in FIGS. 3-4, the first membrane 30 has an outer surface 32, an inner surface 33, and a perimeter 34. Similarly, the second membrane 40 has an outer surface 42, an inner surface 43, and a perimeter 44. The first and second membranes 30 and 40 may be in sealing engagement with at least one strut segment of the stent 21 to form the membrane pocket 50, as explained in further detail below.

In the exemplary “Palmaz” type stent 21 of FIGS. 1-4, each of the diamond-shaped segments formed between the intersections 27 comprises four strut segments 25a, 25b, 26a and 26b, as depicted in FIG. 3. The two strut segments 25a and 25b are generally parallel to one another, while the other two strut segments 26a and 26b are generally parallel to one another and intersect with the segments 25a and 25b.

In the embodiment of FIGS. 1-4, the first membrane 30 may have multiple regions that are in sealing engagement with multiple strut segments of the stent 21. For example, a first region of the first membrane 30 may be in sealing engagement with the strut segment 25a, a second region of the first membrane 30 may be in sealing engagement with the strut segment 25b, a third region of the first membrane 30 may be in sealing engagement with the strut segment 26a, and a fourth region of the first membrane 30 may be in sealing engagement with the strut segment 26b, as generally shown in FIG. 3.

The four regions of the first membrane 30 that are in sealing engagement with the four strut segments 25a, 25b, 26a and 26b may comprise the four boundaries along the perimeter 34 of the first membrane 30, wherein the four boundaries form a generally diamond-shape and at least partially overlap with the four strut segments 25a, 25b, 26a and 26b. Attachment points may be formed in the areas of overlap between the boundaries of the first membrane 30 and the four strut segments 25a, 25b, 26a and 26b. Any suitable technique may be used to attach the first membrane 30 to the four strut segments 25a, 25b, 26a and 26b, including but not limited to use of a heat or chemical sealant, adhesive, solder, weld, or mechanical means such as clips and the like. The attachment forms a sealing engagement between the first membrane 30 and the four strut segments 25a, 25b, 26a and 26b, such that no significant amount of the therapeutic agent 52 escapes through gaps between the membrane and the strut segments.

As depicted in FIG. 3, the four boundaries along the perimeter 34 of the first membrane 30 partially overlap with the upper surfaces 28 of the four strut segments 25a, 25b, 26a and 26b. However, in other embodiments, one or more of the boundaries of the first membrane 30 may extend over the entirety of the upper surfaces 28 and around towards the lower surfaces 29 of one or more of the strut segments 25a, 25b, 26a and 26b. Thus, the first membrane 30, which generally resides above the strut segments 25a, 25b, 26a and 26b, may be secured to one or more regions beneath the strut segments 25a, 25b, 26a and 26b, or alternatively, around the intersections 27 or another suitable location.

Moreover, the first membrane 30 need not be fixedly attached to all four strut segments 25a, 25b, 26a and 26b. For example, a portion of the perimeter 34 of the first membrane 30 may be tightly wrapped around one or more of the strut segments 25a, 25b, 26a and 26b, without being directly attached to the segments. If tightly wrapped, the membrane may still form a substantially fluid-tight seal with the associated strut segments. In one example, the first membrane 30 may be wrapped around one or more of the strut segments and then at least partially overlap with itself or the second membrane 40, and then two overlapping membrane portions may be adhered or mechanically coupled together.

In FIGS. 1-4, the second membrane 40 may be coupled to one or more of the strut segments 25a, 25b, 26a and 26b in a manner similar to the first membrane 30, as described above. Preferably, the four boundaries along the perimeter 44 of the second membrane 40 at least partially overlap with the lower surfaces 29 of the four strut segments 25a, 25b, 26a and 26b, as depicted in FIG. 4. Accordingly, in the exemplary embodiment shown, the first and second membranes 30 and 40 comprise generally identical diamond-shaped perimeters, and are attached to the upper and lower surfaces 28 and 29, respectively, of the four strut segments 25a, 25b, 26a and 26b. The membrane pocket 50 therefore is formed between the first and second membranes 30 and 40, as shown in FIG. 4.

The therapeutic agent 52, along with a hydrogel or suspension media 54, is provided within the membrane pocket 50, as depicted in FIG. 4. Due to the sealing engagement between the membranes 30 and 40 and the strut segments 25a, 25b, 26a and 26b, as described above, the therapeutic agent 52 is securely retained within the membrane pocket 50. Notably, the membrane pocket 50 is disposed substantially between multiple strut segments of the stent 21, and therefore, a greater therapeutic agent delivery medium may be provided, relative to delivery of the therapeutic agent via the surface area of the stent struts or within a lumen of the struts.

The first and/or second membranes 30 and 40 may comprise a porous material that is designed to release the therapeutic agent 52 over a predetermined period of time. Further, one or both of the first and second membranes 30 and 40 may comprise biodegradable features that, when degraded over time, release the therapeutic agent 52. Solely by way of example, and without limitation, exemplary non-degradable membrane materials may comprise polyurethane, nylon, cellulose, polycarbonate, polyethersulfone, PTFE, and polyvinylidene fluoride, while exemplary degradable membrane materials may comprise PLA, PGA, PLGA, zein, and polyanhydride. The biodegradability of the first and/or second membranes 30 and 40 does not affect the structural integrity of the stent 21 to maintain patency within the bodily passageway.

Notably, when the stent 21 is in the expanded deployed state shown in FIG. 2, the outer surface 32 of the first membrane 30 is adjacent to an inner wall of the bodily passageway, e.g., adjacent to a stenosis within a vessel. Thus, delivery of a therapeutic agent 52 through the first membrane 30 may facilitate delivery in close proximity to the inner wall of the passageway.

Furthermore, when the stent 21 is in the expanded deployed state shown in FIG. 2, the outer surface 42 of the second membrane 40 faces inwards toward a lumen of the bodily passageway. Thus, delivery of a therapeutic agent 52 through the second membrane 40 may facilitate delivery directly into flow occurring in the lumen.

In one embodiment, the inner and outer membranes 30 and 40 may have different characteristics relative to one another. For example, if it is desirable to quickly deliver a first therapeutic agent to a vessel wall, the outer surface 32 of the first membrane 30 of the membrane pocket 50 may comprise a greater porosity relative to the second membrane 40.

Additionally, the system 20 may comprise multiple membrane pockets at different locations, which are capable of delivering different therapeutic agents and releasing the agents in different stages. For example, as shown in FIGS. 1-2, a second membrane pocket 50′ is provided at a location distal to, and circumferentially offset from, the first membrane pocket 50. The second membrane pocket 50′ may deliver the same or a different therapeutic agent relative to the first membrane pocket 50, and the delivery of the agent may occur at a faster, slower, or identical rate relative to the first membrane pocket 50. Moreover, the first membrane pocket 50 may deliver the associated agent in an inward direction towards the vessel lumen via the second membrane 40 while the second membrane pocket 50′ delivers its associated agent in an outward direction towards the vessel wall via the first membrane 30′, or vice versa. Alternatively or additionally, both membrane pockets 50 and 50′ may deliver their respective therapeutic agents towards a vessel wall or both may deliver the agents towards a vessel lumen. Moreover, it will be understood that while two exemplary pockets 50 and 50′ are shown in FIGS. 1-2, the system 20 may comprise any number of pockets in any of the spaces between struts of the stent 21.

Advantageously, the system 20 may enhance delivery of multiple different therapeutic agents to a desired target site. Moreover, the quantity of the therapeutic agents delivered is not limited by the surface area of the stent struts or the lumen diameter of the struts. Rather, a significantly enhanced quantity of one or more agents may be delivered generally in membrane pockets disposed between multiple stent struts. Therefore, it is not necessary to increase the surface area of the stent struts or the lumen diameter of the struts to deliver a greater quantity of one or more therapeutic agents, and consequently the stent profile and flexibility is not compromised.

The therapeutic agents used in conjunction with the system 20, and any of the other systems described below, may be chosen to perform a desired function upon release from the membrane pockets 50 and 50′, and may be tailored for use based on the particular medical application. For example, the therapeutic agent can be selected to treat indications such as coronary artery angioplasty, renal artery angioplasty, carotid artery surgery, renal dialysis fistulae stenosis, or vascular graft stenosis. The therapeutic agent may be delivered in any suitable medium. The therapeutic agent may be selected to perform one or more desired biological functions, for example, promoting the ingrowth of tissue from the interior wall of a body vessel, or alternatively, to mitigate or prevent undesired conditions in the vessel wall, such as restenosis. Many other types of therapeutic agents may be used in conjunction with the system 20.

The therapeutic agent employed also may comprise an antithrombogenic bioactive agent, e.g., any bioactive agent that inhibits or prevents thrombus formation within a body vessel. Types of antithrombotic bioactive agents include anticoagulants, antiplatelets, and fibrinolytics. Anticoagulants are bioactive materials which act on any of the factors, cofactors, activated factors, or activated cofactors in the biochemical cascade and inhibit the synthesis of fibrin. Antiplatelet bioactive agents inhibit the adhesion, activation, and aggregation of platelets, which are key components of thrombi and play an important role in thrombosis. Fibrinolytic bioactive agents enhance the fibrinolytic cascade or otherwise aid in dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa and phosphodiesterase inhibitors; and fibrinolytics such as plasminogen activators, thrombin activatable fibrinolysis inhibitor (TAFI) inhibitors, and other enzymes which cleave fibrin.

Additionally, or alternatively, the therapeutic agents may include thrombolytic agents used to dissolve blood clots that may adversely affect blood flow in body vessels. A thrombolytic agent is any therapeutic agent that either digests fibrin fibers directly or activates the natural mechanisms for doing so. Examples of commercial thrombolytics, with the corresponding active agent in parenthesis, include, but are not limited to, Abbokinase (urokinase), Abbokinase Open-Cath (urokinase), Activase (alteplase, recombinant), Eminase (anitstreplase), Retavase (reteplase, recombinant), and Streptase (streptokinase). Other commonly used names are anisoylated plasminogen-streptokinase activator complex; APSAC; tissue-type plasminogen activator (recombinant); t-PA; rt-PA. While a few exemplary therapeutic agents have been listed, it will be apparent that numerous other suitable therapeutic agents may be used in conjunction with the system 20 and delivered via the membrane pockets 50 and 50′ to perform various biological functions.

Referring now to FIG. 5, in one embodiment, the first membrane 30 may comprise a corrugated material having a series of parallel grooves 36 to facilitate movement of the membrane between the contracted to expanded states of FIGS. 1-2, respectively. The first membrane 30 also may comprise an elastic or compliant material, with or without corrugations. Similarly, the second membrane 40 may comprise a corrugated, stretchable and/or compliant material. In this manner, the first and second membranes 30 and 40 will not significantly adversely impact the structural characteristics of the stent 21 during expansion.

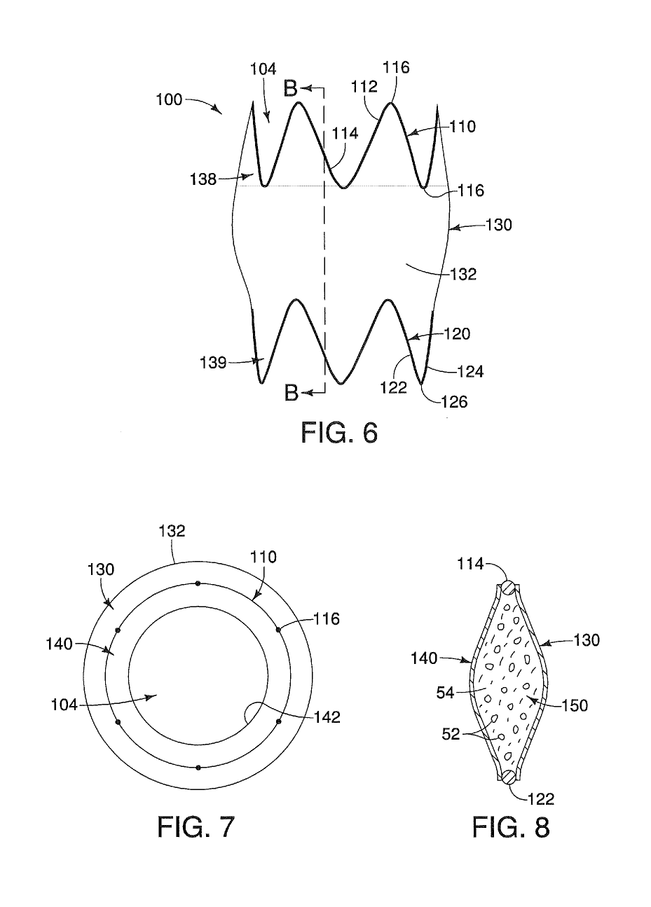

Referring now to FIGS. 6-8, another embodiment of apparatus 100 for delivering at least one therapeutic agent is shown. The system 100 comprises a first stent 110 and a second stent 120. Both the first and second stents 110 and 120 preferably comprises generally zig-zag shapes, commonly referred to as “Z-stents” or “Gianturco stents.” In particular, the first stent 110 may be formed from a single wire comprising a plurality of substantially straight first segments 112 and second segments 114 having a plurality of bent segments 116 disposed therebetween. Similarly, the second 120 may be formed from a single wire comprising a plurality of substantially straight first segments 122 and second segments 124 having a plurality of bent segments 126 disposed therebetween, as shown in FIG. 6.

Each of the first and second stents 110 and 120 may be manufactured from a super-elastic material. Solely by way of example, the super-elastic material may comprise a shape-memory alloy, such as a nickel titanium alloy (Nitinol). If the stents 110 and 120 comprise a self-expanding material such as Nitinol, the stents may be heat-set into the desired expanded configuration, whereby the stents 110 and 120 can assume a relaxed configuration in which it assumes the preconfigured first expanded inner diameter upon application of a certain cold or hot medium. Alternatively, or additionally, the stents 110 and 120 may be made from other metals and alloys that allow the stents 110 and 120 to return to their original, expanded configuration upon deployment, without inducing a permanent strain on the material due to compression. Solely by way of example, the stents 110 and 120 may comprise other materials such as stainless steel, cobalt-chrome alloys, amorphous metals, tantalum, platinum, gold and titanium. The stents 110 and 120 also may be made from non-metallic materials, such as thermoplastics and other polymers.

In the embodiment of FIGS. 6-8, a first membrane 130, second membrane 140, and membrane pocket 150 are similar to the first membrane 30, second membrane 40, and membrane pocket 50, respectively, as explained with respect to the embodiment of FIGS. 1-4 above. However, in the embodiment of FIGS. 6-8, the first and second membranes 130 and 140 are disposed between the longitudinally spaced apart first and second stents 110 and 120.

Proximal and distal regions of the first membrane 130 may be in sealing engagement with multiple strut segments of the first and second stents 110 and 120, respectively. Preferably, a proximal region 138 of the first membrane 130 is shaped to match a pattern of the first zig-zag stent 110, while a distal region 139 of the first membrane 130 is shaped to match a pattern of the second zig-zag stent 120, as depicted in FIG. 6. Proximal attachment points may be formed in the areas of overlap between the proximal region 138 of the first membrane 130 and the strut segments 112, 114 and 116 of the first stent 110. Any suitable technique may be used to attach the proximal region 138 of the first membrane 130 to the strut segments 112, 114 and 116, including but not limited to use of a heat or chemical sealant, adhesive, solder, weld, or mechanical means such as clips and the like. The attachment forms a sealing engagement between the first membrane 130 and the strut segments of the stent 110, such that no significant amount of the therapeutic agent 52 escapes through gaps between the membrane and the strut segments. In a similar manner, the distal region 139 of the first membrane 130 may be in sealing engagement with the strut segments 122, 124 and 126 of the second stent 120, as shown in FIG. 6.

Similarly, proximal and distal regions of the second membrane 140 also may be in sealing engagement with the first and second stents 110 and 120, respectively, thereby forming the membrane pocket 150 between the first and second membranes 130 and 140. As shown in FIG. 8 and generally described above, the therapeutic agent 52, along with a hydrogel or suspension media 54, is provided within the membrane pocket 150.

Further, as shown in FIGS. 6-7, an outer surface 132 of the first membrane 130 therefore may be positioned adjacent to an inner wall of a bodily passageway when the stents 110 and 120 are in the expanded state, and the inner surface 143 of the second membrane 140 may be positioned adjacent to an inner lumen of the passageway. As noted above, the inner and outer membranes 130 and 140 may have different characteristics relative to one another, including but not limited to porosity of the material. It should also be noted that a lumen 104 maintains blood flow through the system 100 when the first and second stents 110 and 120 are in the expanded state, as depicted in FIGS. 6-7.

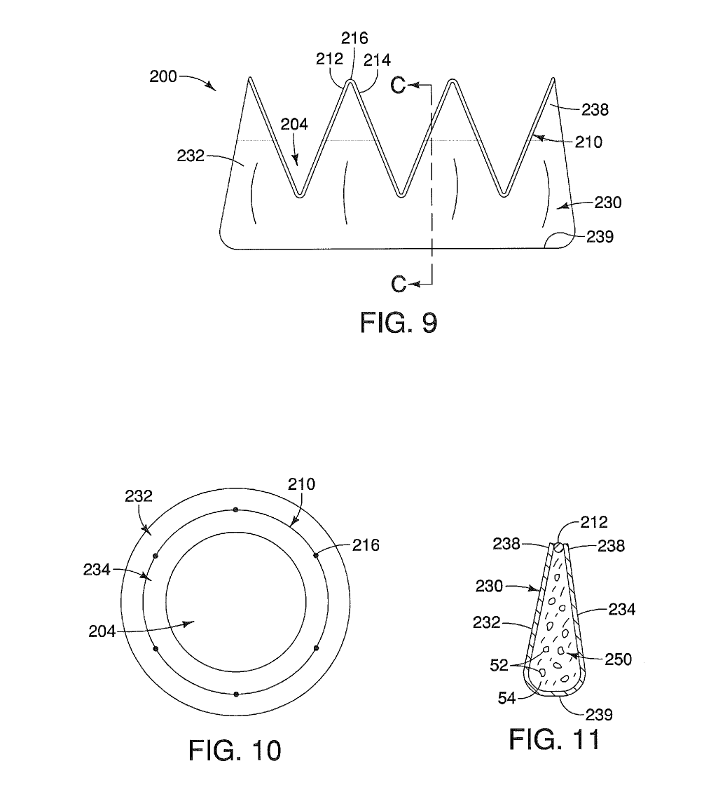

Advantageously, like the embodiment of FIGS. 1-4 above, in the embodiment of is FIGS. 6-8 the quantity of the therapeutic agent 52 delivered is not limited by the surface area of the stent struts or the lumen diameter of the struts. Rather, the size of the membrane pocket 150 is determined, in part, based on the longitudinal spacing of the first and second stents 110 and 120. Accordingly, a significantly enhanced quantity of the therapeutic agent 50 may be delivered via the membrane pocket 150. Referring now to FIGS. 9-11, another embodiment of system or apparatus 200 for delivering at least one therapeutic agent is shown. The system 200 comprises a single zig-zag stent 210 that may be identical to the first and second zig-zag stents 110 and 120 described above, and comprises a plurality of substantially straight first segments 212 and second segments 214 having a plurality of bent segments 216 disposed therebetween. In the embodiment of FIGS. 9-11, only a single membrane 230 is provided, wherein the membrane 230 is shaped to form a membrane pocket 250 therein, as shown in FIG. 11.

The membrane 230, when folded into the configuration shown in FIG. 11, forms first and second regions 232 and 234 having a region 239 disposed therebetween. When coupled to the stent 210, the first region 232 effectively faces radially outward, the second region 234 effectively faces radially inward, and the region 239 forms a distal end of the system 200, as depicted in FIGS. 9-11. The end regions 238 of the first membrane 230 form proximal attachment points that permit attachment of the first membrane 230 to the struts of the stent 210, as best seen in FIG. 11. Any suitable technique may be used to attach the end regions 238 of the membrane 230 to the strut segments 112, 114 and 116, including but not limited to use of a heat or chemical sealant, adhesive, solder, weld, or mechanical means such as clips and the like, as described above.

Accordingly, when assembled, the proximal boundaries of the pocket 250 are defined by the attachment points of the membrane 230 to struts of the stent 210, while the distal boundary of the pocket 250 is defined by the region 239, and the outer and inner boundaries of the pocket 250 are defined by the first and second regions 232 and 234, respectively. The membrane pocket 250 therefore forms a generally tear-drop shape, as shown in FIG. 11. It should be noted that a lumen 204 maintains blood flow through the system 200 when the stents 210 is in the expanded state, as depicted in FIGS. 9-10.

Preferably, the attachment of the membrane 230 to the stent 210 is designed in such a manner to reduce radially inward sagging of the distal region 239 of the pocket 250, which in effect is not supported by a stent strut. Reducing the longitudinal length of the pocket 250, i.e., by reducing the length of the first and second regions 232 and 234, may increase the tautness of the pocket 250 and maintain the size of the lumen 204.

Advantageously, like the embodiment of FIGS. 1-4 and 6-8 above, in the embodiment of FIGS. 9-11 the quantity of the therapeutic agent 52 delivered is not limited by the surface area of the stent struts or the lumen diameter of the struts. Rather, the size of the membrane pocket 250 is determined, in part, based on the length of the membrane 230. Accordingly, a significantly enhanced quantity of the therapeutic agent 50 may be delivered in the membrane pocket 250.

While the above-described embodiments have illustrated use of one or more membrane pockets with Palmaz-type stents and Gianturco-type stents, it will be appreciated that numerous other stent designs may be used. Generally, nearly any stent design may be used with the membrane pockets described herein, so long as one or more of the membranes may sealingly engage one or more struts to form an enclosed membrane pocket between struts that is capable of delivering a therapeutic agent.

It is to be appreciated that the features of the various embodiments described herein may be combined with one another, for example to produce a stent having a variety of different types of pocket or pouch structures. Similarly, although the preferred embodiments have been described in connection with a stent, the teachings herein are equally applicable to other types of medical devices, including but not limited to stent grafts, vena cava filters, occluders and so on.

While various embodiments of the invention have been described, the invention is not to be restricted thereto. Moreover, the advantages described herein are not necessarily the only advantages of the invention and it is not necessarily expected that every embodiment of the invention will achieve all of the advantages described.

The disclosures in U.S. patent application No. 61/181,956, from which this application claims priority, and in the abstract accompanying this application are hereby incorporated by reference.

1-20. (canceled)

21. Apparatus for delivering at least one therapeutic agent to a target site, comprising:
at least one stent having first and second strut segments;
a first membrane having a first region in sealing engagement with the first strut segment, and a second region in sealing engagement with the second strut segment;
a second membrane having a first region in sealing engagement with the first strut segment, and a second region in sealing engagement with the second strut segment;
a first membrane pocket disposed between the first and second membranes; and
a first therapeutic agent disposed within the first membrane pocket.

22. The apparatus of claim 21 where the stent comprises a plurality of generally diamond-shaped segments, each diamond-shaped segment formed by four strut segments, and where the first membrane is in sealing engagement with upper surfaces of each of the four strut segments.

23. The apparatus of claim 22 where the second membrane is in sealing engagement with lower surfaces of each of the four strut segments.

24. The apparatus of claim 21 where at least one of the first and second membranes comprises a material designed to release the therapeutic agent over a predetermined period of time.

25. The apparatus of claim 21 where, when the stent is in an expanded state in a bodily passageway, the first membrane is positioned adjacent to an inner wall of a bodily passageway and the second membrane is positioned adjacent to a lumen of the bodily passageway.

26. The apparatus of claim 25 where the first and second membranes comprise different characteristics relative to one another.

27. The apparatus of claim 21 further comprising:
a second membrane pocket, separate and discrete from the first membrane pocket; and
a second therapeutic agent, different from the first therapeutic agent, disposed within the second membrane pocket.

28. The apparatus of claim 21 where at least one of the first and second membranes comprises a corrugated material.

29. The apparatus of claim 21 further comprising:
first and second stents that are longitudinally spaced apart from one another,
where the first membrane has a proximal region sealingly engaging strut segments of the first stent and a distal region sealingly engaging strut segments of the second stent, and
where the second membrane has a proximal region sealingly engaging strut segments of the first stent and a distal region sealingly engaging strut segments of the second stent.

30. The apparatus of claim 29 where at least one of the first and second stents comprises a zig-zag shape.

31. A method for delivering at least one therapeutic agent to a target site, comprising:
providing at least one stent having first and second strut segments;
sealingly engaging a first region of a first membrane to the first strut segment, and further sealingly engaging a second region of the first membrane to the second strut segment;
sealingly engaging a first region of a second membrane to the first strut segment, and further sealingly engaging a second region of the second membrane to the second strut segment, thereby forming a first membrane pocket between the first and second membranes; and
delivering a first therapeutic agent to the target site via the first membrane pocket.

32. The method of claim 31 where the stent comprises a plurality of generally diamond-shaped segments, each diamond-shaped segment formed by four strut segments, the method further comprising:
sealingly engaging the first membrane with upper surfaces of each of the four strut segments; and
sealingly engaging the second membrane with lower surfaces of each of the four strut segments.

33. The method of claim 31 where, when the stent is in an expanded state, the first membrane is positioned adjacent to an inner wall of a bodily passageway and the second membrane is positioned adjacent to a lumen of the bodily passageway.

34. The method of claim 31 further comprising:
providing a second membrane pocket, separate and discrete from the first membrane pocket; and
delivering a second therapeutic agent, different from the first therapeutic agent, via the second membrane pocket.

35. The method of claim 31 further comprising:
providing first and second stents that are longitudinally spaced apart from one another;
sealingly engaging a proximal region of the first membrane to strut segments of the first stent and sealingly engaging a distal region of the first membrane to strut segments of the second stent; and
sealingly engaging a proximal region of the second membrane to strut segments of the first stent and sealingly engaging a distal region of the second membrane to strut segments of the second stent.

36. The method of claim 35 where at least one of the first and second stents comprises a zig-zag shape.

37. Apparatus for delivering at least one therapeutic agent to a target site, comprising:
a stent having a plurality of strut segments;
a membrane that is folded over to form a membrane pocket, where the membrane pocket comprises at least two proximal boundaries, a distal boundary, and first and second regions formed therebetween, where the two proximal boundaries are in sealing engagement with at least one of the strut segments of the stent; and
a therapeutic agent disposed within the membrane pocket.

38. The apparatus of claim 37 where the membrane pocket forms a generally tear-drop shape.

39. The apparatus of claim 37 where the stent comprises a zig-zag shape.

40. The apparatus of claim 37 where, when the stent is in an expanded state, the first region of the membrane pocket is positioned adjacent to an inner wall of a bodily passageway and the second region of the membrane pocket is positioned adjacent to a lumen of the bodily passageway.
